(19) United States
(12) Patent Application Publication
(10) Pub. No.: US 2013/0030281 A1
(43) Pub. Date: Jan. 31, 2013

(54) Hashimotos Thyroiditis Detection and Monitoring

(71) Applicant: Jasjit S Suri, Roseville, CA (US)

(72) Inventor: Jasjit S Suri, Roseville, CA (US)

(21) Appl. No.: 13/626,487

(22) Filed: Sep. 25, 2012

Related U.S. Application Data
(63) Continuation-in-part of application No. 12/799,177, filed on Apr. 20, 2010.
Application No. 12/799,177 is a continuation-in-part of application No. 12/802,431, filed on Jun. 7, 2010, now Pat. No. 8,313,437.
Application No. 12/802,431 is a continuation-in-part of application No. 12/896,875, filed on Oct. 2, 2010.
Application No. 12/896,875 is a continuation-in-part of application No. 12/960,491, filed on Dec. 4, 2010.
Application No. 12/960,491 is a continuation-in-part of application No. 13/053,971, filed on Mar. 22, 2011.
Application No. 13/053,971 is a continuation-in-part of application No. 13/077,631, filed on Mar. 31, 2011.
Application No. 13/077,631 is a continuation-in-part of application No. 13/107,935, filed on May 15, 2011.
Application No. 13/107,935 is a continuation-in-part of application No. 13/219,695, filed on Aug. 28, 2011.
Application No. 13/219,695 is a continuation-in-part of application No. 13/253,952, filed on Oct. 5, 2011.
Application No. 13/253,952 is a continuation-in-part of application No. 13/407,602, filed on Feb. 28, 2012.
Application No. 13/407,602 is a continuation-in-part of application No. 13/412,118, filed on Mar. 5, 2012.
Application No. 13/412,118 is a continuation-in-part of application No. 13/449,518, filed on Apr. 18, 2012.
Application No. 13/449,518 is a continuation-in-part of application No. 13/465,091, filed on May 7, 2012.
Application No. 13/465,091 is a continuation-in-part of application No. 13/589,802, filed on Aug. 20, 2012.

(51) Int. Cl. A61B 6/00; A61B 5/055; A61B 6/03; A61B 8/00

(52) U.S. Cl. 600/410; 600/407; 600/437; 600/425

(57) ABSTRACT

Hashimoto's Thyroiditis (HT) is the most common type of inflammation of the thyroid gland and accurate diagnosis of HT would be advantageous in predicting thyroid failure. The application presents a three tier architecture for image-based diagnosis and a monitoring application using a network cloud. The presentation layer is run on the tablet (e.g., a mobile device), while the business and persistence layers run on a single network cloud or distributed on different network clouds in a multi-tenancy and multi-user application. Such three tier architecture is used for automated data mining application for diagnosis of Hashimoto's Thyroiditis (HT) Disease using ultrasound.

### PRIORITY APPLICATIONS

This is a continuation-in-part patent application of co-pending patent application Ser. No. 12/799,177; filed Apr. 20, 2010 by the same applicant. This is also a continuation-in-part patent application of co-pending patent application Ser. No. 12/802,431; flied Jun. 7, 2010 by the same applicant. This is also a continuation-in-part patent application of co-pending patent application Ser. No. 12/896,875; filed Oct. 2, 2010 by the same applicant. This is also a continuation-in-part patent application of co-pending patent application Ser. No. 12/960,491; filed Dec. 4, 2010 by the same applicant. This is also to continuation-in-part patent application of co-pending patent application Ser. No. 13/053,971; filed Mar. 22, 2011 by the same applicant. This is also a continuation-in-part patent application of co-pending patent application Ser. No. 13/077,631; filed Mar. 31, 2011 by the same applicant. This is also a continuation-in-part patent application of co-pending patent application Ser. No. 13/107,935; filed May 15, 2011 by the same applicant. This is also as continuation-in-part patent application of co-pending patent application, Ser. No. 13/219,695; filed Aug. 28, 2011 by the same applicant. This is also a continuation-in-part patent application of co-pending patent application, serial no, 13/253,952; filed Oct. 5, 2011 by the same applicant. This is also a continuation-in-part patent application of co-pending patent application Ser. No. 13/407,602; filed Feb. 28, 2012 by the same applicant. This is also a continuation-in-part patent application of co-pending patent application Ser. No. 13/412,118; filed. Mar. 5, 2012 by the same applicant. This is also a continuation-in-part patent application of co-pending patent application Ser. No. 13/449,518; filed Apr. 18, 2012 by the same applicant. This is also a continuation-in-part patent application of co-pending patent application Ser. No. 13/465,091; filed May 7, 2012 by the same applicant. This is also a continuation-in-part patent application of co-pending patent application Ser. No. 13/589,802; filed Aug. 20, 2012 by the same applicant. This present patent application draws priority from the referenced co-pending patent applications. The entire disclosures of the referenced co-pending patent applications are considered part of the disclosure of the present application and are hereby incorporated by reference herein in its entirety.

### TECHNICAL FIELD

This application relates to a method and system for use with data processing and imaging systems, according to one embodiment, and more specifically, for a mobile architecture using cloud for data mining application such as Hashimoto Thyroiditis (HT) classification and diagnosis.

### BACKGROUND

Imaging-based technologies have been active for over a century and today the same imaging-based technologies are used electronically for creating pictures of the human body and examining it. Majority of these imaging modalities are non-invasive and painless. Depending upon the symptoms of the patient's disease, a physician will choose a type of the imaging modality, its diagnosis, treatment and monitoring. Some of the most famous medical imaging modalities are Ultrasound, X-ray, MR, CT, PET, SPECT and now more molecular and cellular level. These imaging modalities are conducted by the radiologist or a technologist who are well trained, to operate and know the safety rules.

The importance of imaging-based techniques for diagnosis, treatment, monitoring is increasing day-by-day. Thus more and more body images are generated every day. Hospitals and health care providers are generating image data at an alarming rate. There is no doubt that one has to design complex medical imaging software for diagnosis, treatment and monitoring, but it is becoming challenging to access these data in this age of the world. Storage of the medial images is one issue and how to access this data for decision making such as diagnosis, treatment and monitoring is another issue.

### BRIEF SUMMARY AND THE OBJECTS OF THE DISCLOSED EMBODIMENTS

Hashimoto's Thyroiditis (HT) is an autoimmune disease that is characterized by lymphocytic infiltration and disruption of thyroid gland tissue architecture and production of specific autoantibodies against thyroid. Hashimoto's Thyroiditis is the most common type of inflammation of the thyroid gland, and a most frequent cause of hypothyroidism. Early diagnosis of Hashimoto's Thyroiditis would be advantageous in predicting thyroid failure.

The following are the commonly first lowed diagnostic criteria of Hashimoto's Thyroiditis: (i) a positive test for thyroid autoantibodies in serum, (ii) an elevated serum thyrotropin (TSH) concentration, or (ii) the presence of lymphocytic infiltration of the thyroid in histopathologic examination. Other common diagnostic tests are fine-needle aspiration biopsy and an ultrasound (US) scan. Among these techniques, the most preferred choice is thyroid ultrasonography which is a non-invasive diagnostic test that provides an image of the structure and the characteristics of thyroid. It was reported that autoimmune thyroiditis could be successfully excluded on the basis of ultrasound alone in 1962 cases among 2322 cases studied (84%). Moreover, ultrasound is affordable, widely available, does not use harmful ionizing radiation, and has relatively shorter acquisition time compared to other modalities like Computed Tomography (CT) and Magnetic Resonance Imaging (MRI).

A regular thyroid tissue is characterized by homogeneity and high echogenicity in ultrasound. In Hashimoto's Thyroiditis, the architecture destruction of the follicles and lymphocytic infiltrations result in decreased echogenicity. There is evidence that reduced thyroid echogenicity demonstrated by ultrasonography is a strong predictor of chronic autoimmune thyroiditis even when this disorder has not been suspected clinically. Earlier, this change in echogenicity was evaluated based on a rough visual comparison with the surrounding neck muscular tissue. Subsequently, analysis of grayscale histogram was carried out for quantitative measurement of echogenicity decline. Other studies too have proposed that computerized gray-scale ultrasound gives quantitative determination of thyroid echogenicity and mean tissue density in thyroid autoimmune diseases.

These computerized methods have the advantages of being more objective. However, they are limited by the fact that there is lack of procedure standardization because individual investigators use various initial ultrasound settings. Echogenic appearance of the thyroid gland varies with the adjustment of the gain. Thus, ultrasound diagnosis of Hashimoto's Thyroiditis is still operator-dependent and defined conditions are necessary to evaluate exact data. To compensate the attenuation of ultrasound energy as the pulses traverse the different layers of the neck, a corresponding amplification of ultrasound signals by the operator is necessary. Too much amplification may mask a true reduction in thyroid echogenicity, and too little amplification may lead to a false diagnosis of reduced thyroid echogenicity. Furthermore, in the end stage of Hashimoto's Thyroiditis, mean tissue density assessment may be misleading because of the presence of a combination of the hyperechoic and hypoechoic signals in the examined zone. These operator dependent and echogenic limitations is another reason for development of an objective, non-invasive, and accurate Hashimoto's Thyroiditis diagnosis support systems that use medical image mining techniques.

Image mining uses techniques from statistics and artificial intelligence to determine features which quantitatively characterize the patterns in an image. In this context, these features quantify the histopathologic components of the US thyroid images obtained from normal and Hashimoto's Thyroiditis-affected patients. These features can then be used to train supervised learning based classifiers to relate the extracted features from an image to the corresponding class (normal or Hashimoto's Thyroiditis-affected abnormal). The trained classifiers can then be used to predict the class of a new image which was not used for training. The key objective of this work is to develop one such Computer Aided Diagnosis (CAM-based paradigm that uses classification techniques to automatically differentiate ultrasound images from normal and Hashimoto's Thyroiditis affected cases in cloud-based settings. Thus, the proposed technique will have the following characteristics: (a) It will use thyroid images from the most commonly used, affordable and available, non-invasive and safe ultrasound modality; (b) The interpretations will be more objective and reproducible due to the use of standard image analysis algorithms; (c) Use of this technique will, not incur any additional cost because the proposed algorithm can be written into a software application at no extra cost and can be installed in the physician's computer; and (d) It will act as an adjunct tool that provides to second opinion on the initial diagnosis thereby increasing the confidence of the physician in planning, the subsequent treatment evaluation protocol for the patient.

This application is a novel method that presents a three tier architecture for image-based diagnosis and monitoring application using cloud. The presentation layer is run on the tablet (mobile device), while the business and persistence layer runs on the cloud or as set of clouds. The business and presentation layers can be in one cloud or multiple clouds. Further, the system can accommodate multiple users in this architecture set-up with multiple tenancies.

The application is designed to assist the endocrinologist, internal medicine or a physician in examining the Thyroid Disease and in particular diagnosis the Hashimoto Disease.

Data access from remote locations has become important day-by-day in this high information technology world. Due to this, now Cloud-based imaging can provide solution to such challenges. Even though, HIPPA or security or data ownership technologies are evolving, but the pros of Cloud-based technologies have outweighed the cons.

The Cloud-based technology offers, the first one is pricing. Cloud-based processing is less expensive due to low storage cost. Additional benefit is that if one uses Cloud for Software as a Service (SaaS) application, the storage cost can be free.

Another advantage of Cloud-based processing is the capacity to handle. Compared to costs for the local processing when the data storage requirements are changing dynamically, Cloud-based capacity may be advantageous. Expansion possibility is easy to handle. Emergency storage requirements may also less challenging to handle in Cloud-based processing.

Another major advantage is the disaster recovery. One needs regular backups and maintenance; this can be avoided in the Cloud-based processing.

Having discussed the benefits of Cloud-based processing, it is thus important on how to use Cloud-based services for applications which short time to run applications. This innovative application is about the architecture is designed for medical imaging applications, such as cardiovascular, prostate cancer, ovarian cancer, liver cancer, thyroid cancer and in particular diagnosis of Hashimoto Disease. Today's medical based applications do not just require viewing of the images, but also processing business layers for doctors to get the clinical information such as diagnosis, treatment support and monitoring. Thus the main requirement in today's Cloud-based processing is how to build medical imaging architectures which can benefit from Cloud-based processing, particularly for Thyroid Disease Diagnosis and in particular Hashimoto Disease.

Now that hand held devices have come into the world such as iPad, Samsung tablets or iPhones, it is thus important to understand how to build medical imaging architectures which has several tiers or layers in their architectural designs. This innovative application demonstrates an imaging-based architecture utilizing the Cloud-based processing. The application shows coverage for Thyroid Cancer Diagnosis and in particular Hashimoto Disease. Besides this, the application can be extended to vascular imaging or Cardiac imaging, gynecological imaging, prostate cancer imaging and liver cancer imaging, but is extendable to other anatomies as well.

In view of the foregoing, it is a primary object of the present invention to provide a novel method and apparatus for automated mobile data mining from ultrasound images for diagnostic and monitoring application, particular Hashimoto Disease of Thyroid organ, and further providing extensions to MR or CT images and in general to any other imaging-based data mining application.

It is another object of the present invention to develop a mobile-based architecture which can process images by distributing components of the architecture in different Clouds, but same physical location.

It is another object of the present invention to develop a data mining architecture having the business layer in one Cloud while running the Persistence Layer in another Cloud, not necessarily in the same physical location, particularly applied to the Thyroid Disease Management and in particular for the Hashimoto Disease Diagnosis.

It is another object of the present invention to develop an image-based data mining Cloud-based application which can have multiple-tenants and multiple-users. This data mining application can be where the Business layer is for cardiovascular application (such as IMT measurement, IMTV measurement, Plaque Characterization for Symptomatic vs. Asymptomatic classification of plaque, Stroke Risk computation, and monitoring stroke risk), or urology application such as benign vs. malignant tissue prostate tissue classification for prostate cancer, or gynecological application for classification of ovarian cancer or benign vs. malignant thyroid cancer for endocrinology application, particularly Hashimoto Disease Diagnosis and Classification, or for liver application such as a classification of fatty liver disease (FLD) compared to normal liver.

It is another object of the present invention to provide different configuration options in the Business Layer controlled by the Presentation Layer, where the Presentation Layer can control wirelessly different configurations. Each configuration can be another scientific method for generation of clinical information, such as different set of classifiers used for training and testing during the Thyroid Disease Diagnosis and in particular Hashimoto Disease Diagnosis.

It is another object of the present invention to provide multi-tenancy for data mining applications using distributed architectures, where data mining application can be Business layer for (a) cardiovascular application (such as IMT measurement, IMTV measurement, Plaque Characterization for Symptomatic vs. Asymptomatic classification of plaque, Stroke Risk computation, and monitoring stroke risk); (b) prostate cancer application such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer) and in particular Diagnosis of Thyroid Disease and its management; or (e) classification of liver tissue such as Fatty Liver Disease.

It is another object of the present invention to provide multi-tenancy for data mining applications using, distributed architectures, where multi-tenancy can be using different imaging modality like MRI, CT, Ultrasound or a combination of these for fusion. The multi-tenancy set-up has data mining application where Business layer is: a) cardiovascular application (such as IMT measurement, IMTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer) and in particular Hashimoto Disease Management; or (e) classification of liver tissue such as Fatty Liver Disease.

It is another object of the present invention to provide data mining applications using distributed architectures, where the presentation layer can be hand-held device like iPhone, iPad, Samsung Tablet or notebook or laptop or desktop and data mining application can be for (for (a) cardiovascular application (such as IMT measurement, IMTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer) and in particular Hashimoto Disease Diagnosis and Management or (e) classification of liver tissue such as Fatty Liver Disease.

It is another object of the present invention to provide data mining applications where Business layer for (a) cardiovascular application (such as NT measurement, IMTV measurement. Plaque Characterization for Symptomatic vs. Asymptomatic classification of plaque. Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); and in particular Hashimoto Disease Management or (e) classification of liver tissue such as Fatty Liver Disease, such that it can process the B-mode ultrasound or RF-mode ultrasound image

It is another object of the present invention to provide a method to diagnose a Thyroid Disease, in particular Hashimoto Disease using a combination of training-based image classification, system.

It is another object of the present invention to provide a method to diagnose a Thyroid Disease, in particular Hashimoto, Disease using a combination of training-based image classification system, where the training system (off line system) uses a set of grayscale features such as Entropy features, Gabor wavelet features, Inverse Moment Features, Higher Order Spectra Features.

It is another object of the present invention to provide a method to diagnose a Thyroid Disease, in particular Hashimoto, Disease using a combination of training-based image classification system and testing based image classification system (on line process), where the testing system uses a set of grayscale features such as Entropy features, Gabor wavelet features, Inverse Moment Features and Higher Order Spectra Features.

It is another object of the present invention to provide a method to diagnose a Thyroid Disease, in particular Hashimoto, Disease using a combination of training-based image classification system and testing based image classification system, where the testing system uses a set of grayscale features such as Entropy features, Gabor wavelet features, Inverse Moment Features, Higher Order Spectra Features, such that a feature selection system is able to select the beast combination of features for training and testing classifiers in online and offline processing.

It is another object of the present invention to provide mobile data mining application where Business layer can be a 2D processing unit or a 3D processing unit.

It is another object of the present invention to provide mobile data mining application where Business layer can be a 2D processing unit or a 3D processing unit for diagnostic and monitoring application with different configuration options for the Business Layer.

It is another object of the present invention to provide mobile data mining application where Business layer can be a 2D processing unit or a 3D processing unit for diagnostic and monitoring application with different configuration options for the Business Layer, where these applications use training-based systems.

### BRIEF DESCRIPTION OF THE DRAWINGS

The various embodiments is illustrated by way of example, and not by way of limitation, in the figures of the accompanying drawings in which:

FIG. 1 illustrates an example of mobile architecture system.

FIG. 2 shows an illustrative example of multi-user application using cloud.

FIG. 3 shows an illustrative example of business layer and persistence layer combined on a cloud.

FIG. 4 shows an illustrative example of multi-tenancy approach with business layer and persistence layers in ultrasound framework.

FIG. 5 shows an illustrative example of multi-tenancy approach with business layer and persistence layers in MR framework.

FIG. 6 shows an illustrative example of multi-tenancy approach with business layer and persistence layers in CT framework.

FIG. 7 shows an illustrative example of configuration options from presentation layer for a cloud-based setting.

FIG. 8 shows an illustrative example of multiple clouds demonstrating the components of the applications hosted by different clouds.

FIG. 9 shows an illustrative example of business logic and persistence layers for Hashimoto Disease diagnosis.

FIG. 10 shows an illustrative example of business logic that uses the combination of different feature processors for computing different on-line features.

FIG. 11 shows an illustrative example of business logic that uses the combination of different feature processors using a combination of relative entropy, relative energy, probability of entropy and probability of energy for computing different on-line features

FIG. 12 shows an illustrative example on-line Hashimoto Disease decision making.

FIG. 13 shows the overall view of the system.

FIG. 14 shows a diagrammatic representation of machine in the example form of a computer system within which a set of instructions when executed may cause the machine to perform any one or more of the methodologies discussed herein.

### DETAILED DESCRIPTION OF THE PREFERRED EMBODIMENTS

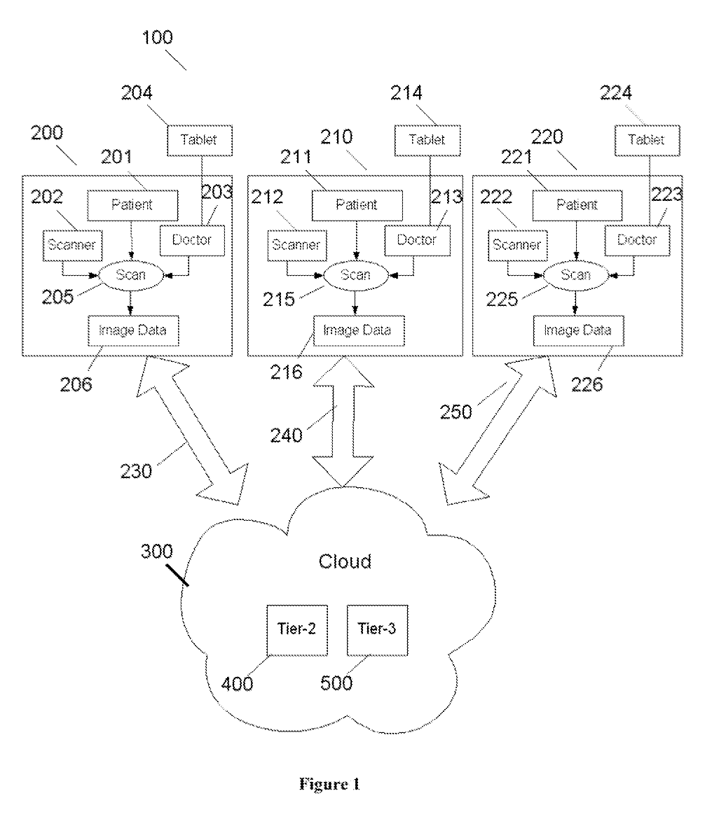

FIG. 1 show the example embodiment 100 of the architecture where the application is split into three tiers: Tier-1 is the presentation layer and Tier-2 and Tier-3 are the business layer and persistence layers. The main advantage of this data mining applications which require large space and still be able to maintain near real-time applications. Another key advantage of such architecture is the ability to decouple business and persistence layers in different clouds and still be able to execute data mining applications. An example embodiment can be for vascular application for atherosclerosis disease monitoring, men's urology application, women's urology application, breast mammography application, liver application, cardiac application, kidney application and thyroid disease application. Blocks 200, 210 and 220 represent different health care systems connected to the cloud 300 having architectures 400 and 500 called as Tier-2 and Tier-3. The connection between the health care systems 200, 210 and 220 to the Cloud 300 is shown using links 230, 240 and 250, respectively. Inside each health care system run the patient data collection systems using the scanners: 205, 215, and 225. These scanners collected image data on the patient 201, 211 and 221 using the scanners 202, 212 and 222, respectively. The physician or technologist is shown in FIG. 203, 213 or 223. The image data collected is shown in the blocks 206, 216 and 226 respectively, which is sent to the cloud 300 using the links 230, 240 and 250, respectively. This application uses automated data mining business layer 401) and persistence layer 500 in the cloud 300. The hand-held devices 204, 214 and 224 (Tier-1) are used for running the data mining applications receding in the cloud 300. These hand-held devices can be iPad or a Tablet or a notebook or a laptop or mobile device. This application can be useful for the architecture for a) cardiovascular application (such as IMT measurement, IMTV measurement, Plaque Characterization for Symptomatic vs. Asymptomatic classification of plaque, Stroke Risk computation, and monitoring, stroke risk) (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease, such that it can process the B-mode ultrasound or RF-mode ultrasound images and (f) thyroid disease classification such as benign thyroid or malignant thyroid or Hashimoto Disease Classification.

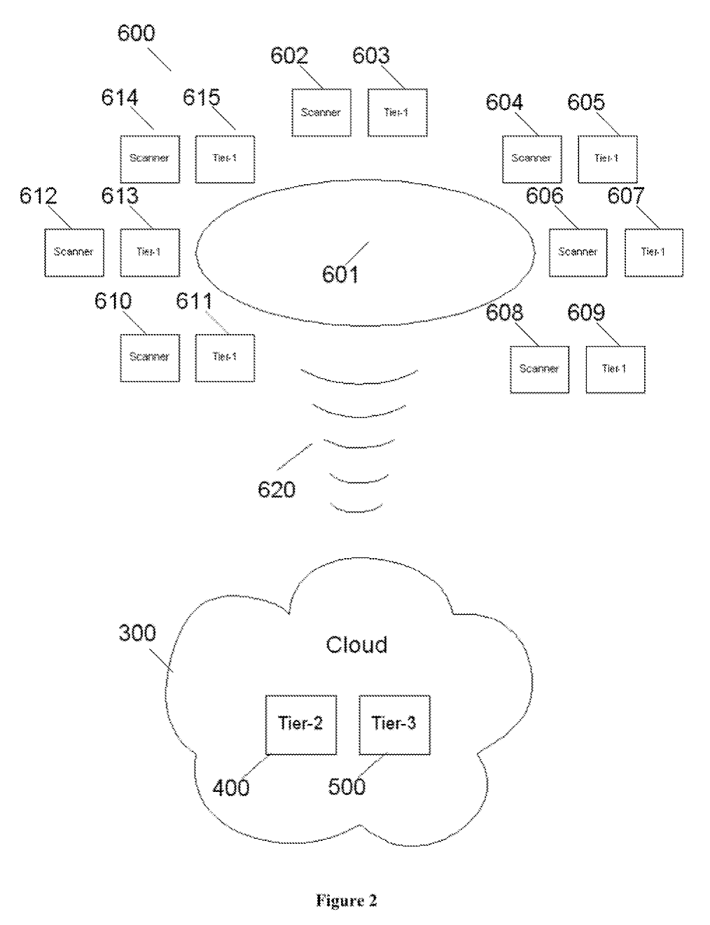

FIG. 2 shows the example embodiment 600 where multiple healthcare providers having multiple Tier-1's and are connected to the Cloud running the Tier-2 and Tier-3. For example 602 and 603 represent one health care system where the Tier-1 block 603 is interacting with the Cloud 300 which has the Tier-2, block 400 and Tier-3, block 500 using a wireless system. Similar pairs can be blocks 604 and 605 representing a scanner and a presentation layer in combination. A cyclic order of such combination representing several healthcare systems can be 606 and 607; 608 and 609; 610 and 611; 612 and 613; 614 and 615, respectively. Those skilled in the art can add more clients in such a cyclic framework. The wireless signals are represented by 620 which are sending the client signals to the Tier-2 which in return can store the intermediate results in Tier-1 Using this architecture, one can also send signal from Tier-1 such as (603, 605, 607, 609, 611,613 and 615) to Tier-3 receding in the Cloud 300. The main advantage of such a system is the decoupling of the Tier-1 from Tier-2 and Tier-3. Those skilled in the art of using client-server model, can reside the Tier-2 on one server and Tier-3 in another server or both Tier-2 and Tier-3 in the same Cloud. Such an application of multi-tenancy can be adapted for a) cardiovascular application (such as IMT measurement, IMTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease, such that it can process the B-mode ultrasound or RF-mode ultrasound images and (f) thyroid disease classification such as benign thyroid or malignant thyroid or Hashimoto Disease Classification, where these applications are the business layers in the three tier architectures.

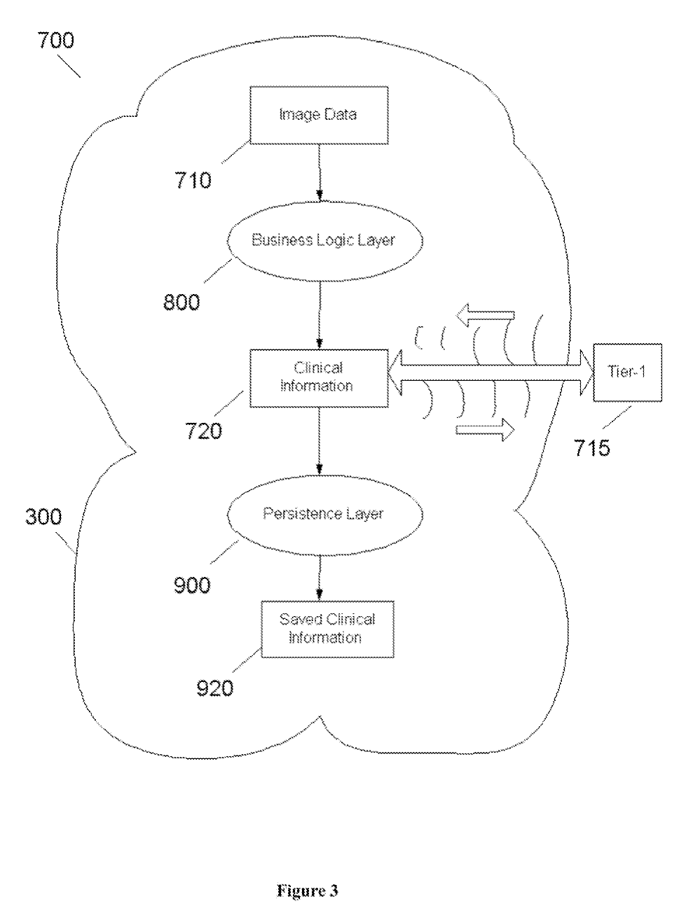

FIG. 3 shows the example embodiment 700, where the Cloud 300 hosts the Business Layer 800 and Persistence Layer 900. The image data is present in the Cloud storage 710. When the Tier-1 presentation layer 715 interacts with the Cloud hosting the application having Tier-2 and Tier-3, then the Clinical information is generated by the Business Logic Layer 800. This Clinical information can be seen on the presentation layer 715. The persistence, layer 900 has the data information which is saved for the application. This can be a database management system which stores the clinical information 920 by running the data mining application. Such a model is very suitable for diagnostic, treatment support and monitoring of the diseases. An example can be for cardiovascular risk application for (a) cardiovascular application (such as IMT measurement, IMTV measurement, Plaque. Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease and (f) thyroid disease classification such as benign thyroid or malignant thyroid or Hashimoto Disease Classification, where these applications are the business layers in the three tier architectures such that it can process the B-mode ultrasound or RF-mode ultrasound images. Under cardiovascular risk, it can compute say the intima-media thickness for the distal wall for the common carotid artery of ultrasound. Along the same lines can be the lumen quantification or lumen segmentation of the common carotid artery ultrasound or any blood vessels. This model is applicable for CCA, brachial artery, aortic arch and peripheral artery. Those skilled in the art can use this application for other arterial systems. Such an application can be for any 2D or 3D application. Another application can be the image data 710 that can be in 3D format and business logic layer 800 can process the image data 710 to give the segmentation results 720 which are being display on the Tier-1 device 710. Those killed in the art can use an iPad, iPhone or Samsung hand held devices for display of the transformed images or segmented images. An example can be a 3D Thyroid image data mining system such as ThyroScan™.

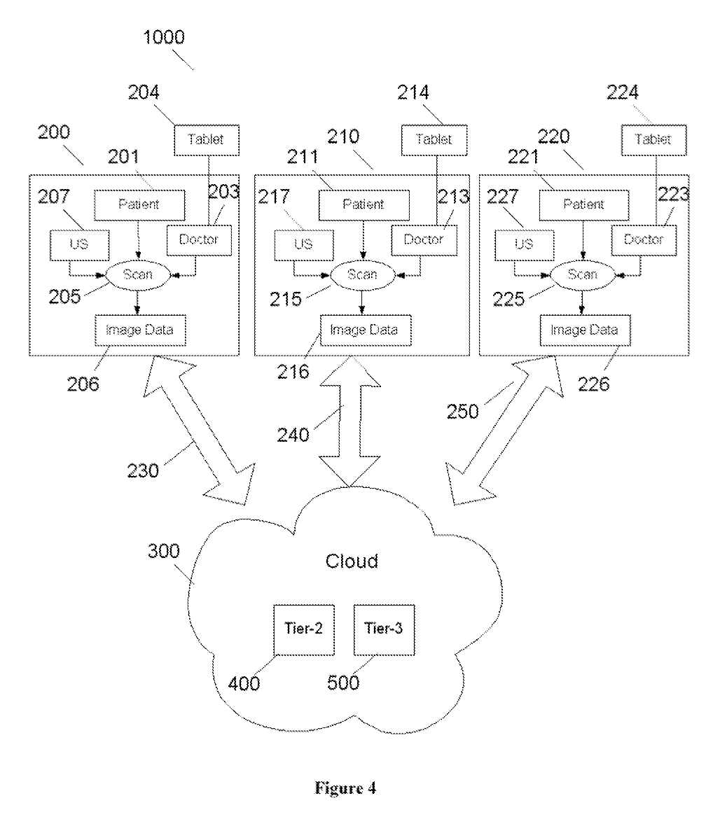

FIG. 4 shows the example embodiment 1000, where the Cloud 300 hosts the Business Layer 400 and Persistence Layer 500. Health care system is represented by blocks 200, 210 and 220. The health care system 200 has the block 207 can be used as a body scanner says an ultrasound scanning system. Similarly, there can be another health care system 210 that has the scanner represented by the block 217. The embodiment 1000 also shows as an example where the third health care system is represented by 220 having the scanner block 227 and is an Ultrasound scanning system. The ultrasound scanner can be a portable ultrasound scanner or an ultrasound scanner having, a cart-based mobile in the hospital or health care system. The embodiment also shows the setup where the patient comes for scanning in the health care system. For example, patient block 201 shows the scanner 207 scanning the patient to generate the image data 206 in the healthcare system 200. Similarly, the embodiment also shows the setup where the patient block 211 shows the scanner 217 scanning the patient to generate the image data 216 in the healthcare system 210. Also shown are the wireless system 230, 240 and 250. Such an set-up can use for (a) cardiovascular application (such as IMT measurement, MTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease; or f) thyroid disease classification such as benign thyroid or malignant thyroid or Hashimoto Disease Classification, where these applications are the business layers in the three tier architectures such that it can process the B-mode ultrasound or RF-mode ultrasound images.

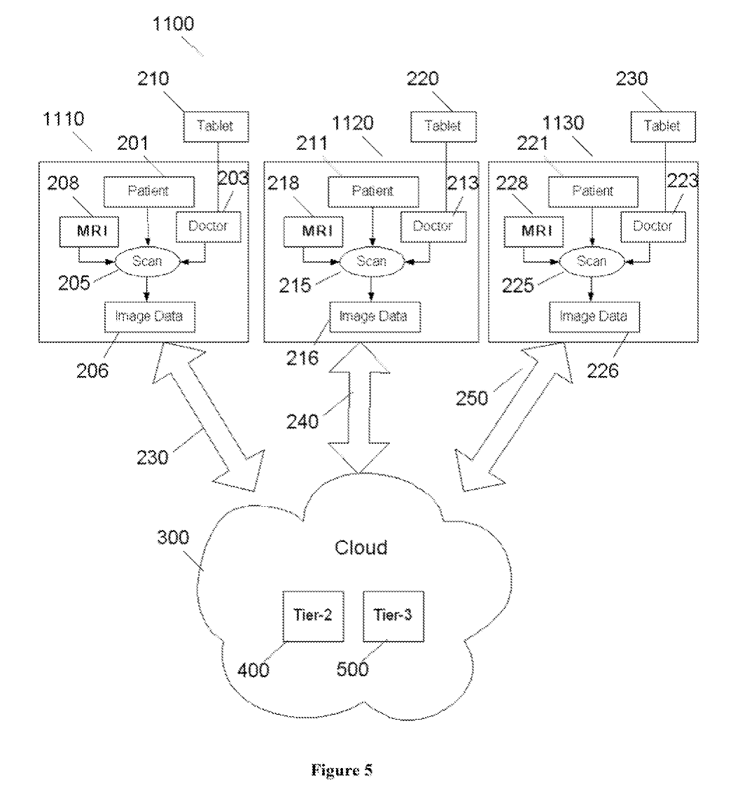

FIG. 5 shows the example embodiment 1100, where multiple tenants 1110, 1120 and 1130 are shown running the data mining application using Cloud 300 which hosts the Business Layer 400 and Persistence Layer 500. Tenant 1110 is the heath care system having the imaging device 208 such as MRI and the technologist or doctor 203 for scanning protocol 205 to yield the image data 206 for the patient 201. Similarly, there is a tenant 1120 is the heath care system having the imaging device 218 such as MRI and the technologist or doctor 213 for scanning protocol 215 to yield the image data 216 for the patient 211. Similarly, there is a tenant 1130 is the heath care system having the imaging device 228 such as MRI and the technologist or doctor 223 for scanning protocol 225 to yield the image data 226 for the patient 221. Also shown are the wireless system 230, 240 and 250. Such an set-up is used for (a) cardiovascular application (such as IMT measurement, IMTV measurement, Plaque Characterization for Symptomatic vs. Asymptomatic classification of plaque, Stroke Risk computation, and monitoring, stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (c) classification of liver tissue such as Fatty Liver Disease or f) thyroid disease classification such as benign thyroid or malignant thyroid or Hashimoto Disease Classification, where these applications are the business layers in the three tier architectures such that it can process MR images.

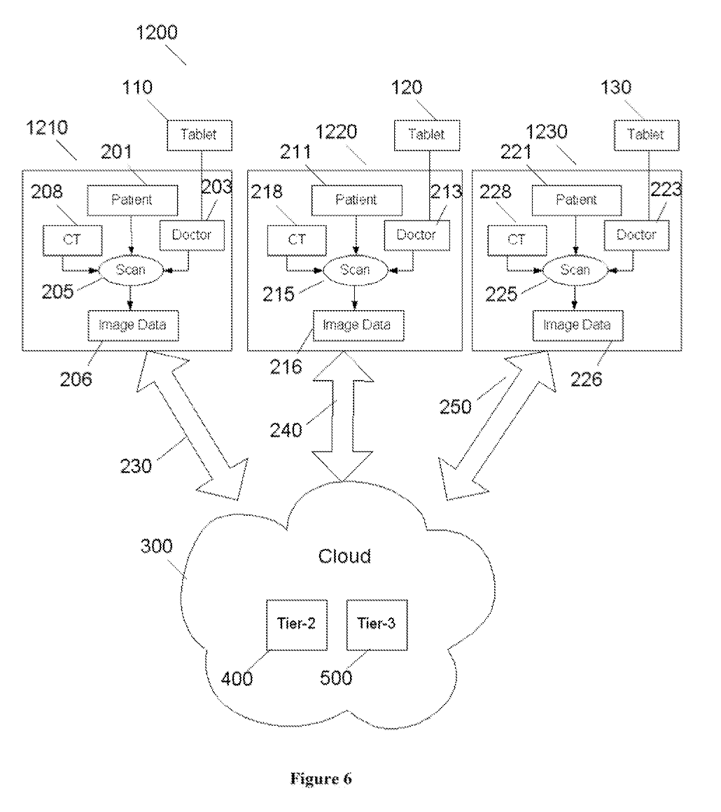

FIG. 6 shows the example embodiment 1200, where multiple tenants 1210, 1220 and 1230 are shown running the data mining application using Cloud 300 which hosts the Business Layer 400 and Persistence Layer 500. Tenant 1210 is the heath care system having the imaging device 208 such as CT and the technologist or doctor 203 for scanning protocol 205 to yield the image data 206 for the patient 201. Similarly, there is a tenant 1220 is the heath care system having the imaging device 218 such as CT and the technologist or doctor 213 for scanning protocol 215 to yield the image data 216 for the patient 211. Similarly, there is a tenant 1230 is the heath care system having the imaging device 228 such as CT and the technologist or doctor 223 for scanning protocol 225 to yield the image data 226 for the patient 221. Also shown are the wireless system 230, 240 and 250. Such an set-up is used for (a) cardiovascular application (such as IMT measurement, IMTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease or f) thyroid disease classification such as benign thyroid or malignant thyroid or Hashimoto Disease Classification, where these applications are the business layers in the three tier architectures such that it can process CT images.

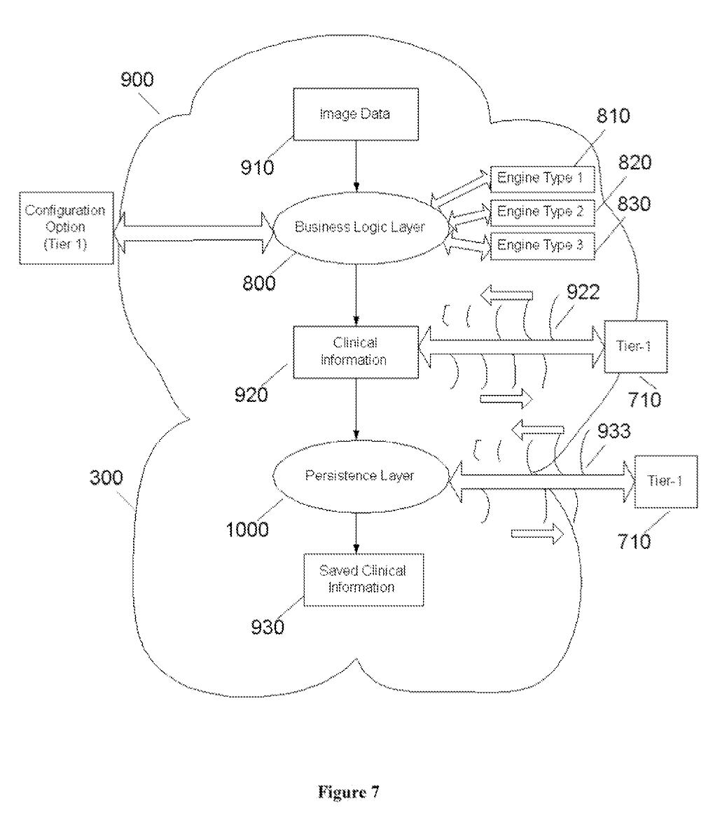

FIG. 7 shows the example embodiment 900 showing different configuration options from presentation layer for a cloud-based setting. Business Logic. Layer 800 received the image data from the tenant using the wireless system, which in turn processes the clinical information and gives the output 920. The configuration option 810, 820 and 830 are available for choosing the different types of engines such as Scientific Engine Type 1, Scientific Engine Type 2 or Scientific Engine Type 3. Such a business layer 800 can be for (a) cardiovascular application (such as IMT measurement, IMTV measurement, Plaque Characterization for Symptomatic vs. Asymptomatic classification of plaque. Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease; or f) thyroid disease classification such as benign thyroid or malignant thyroid or Hashimoto Disease Classification, where these applications are the business layers in the three tier architectures such that it can process the B-mode ultrasound or RF-mode ultrasound images. Tier 1, 710 can interact with the clinical information 920 to display the clinical diagnosis on 710, such as iPhone, iPad, Samsung Table, or even laptop, notebook or Desktop-based display devices. The persistence layer process 1000 processes the clinical information 920 and stores in the persistence layer. This information can also be accessed by Tier-1, 710. Output 930 is the information which is saved in the cloud or local server.

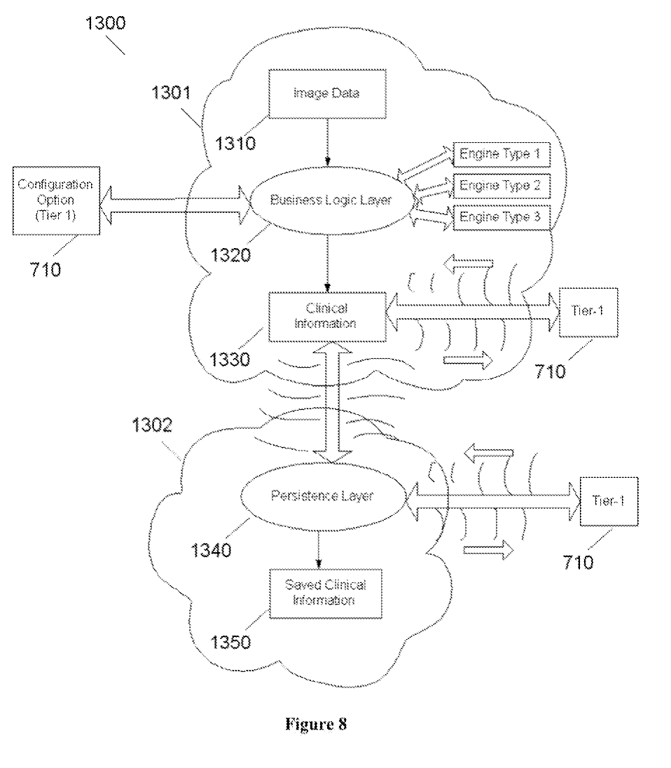

FIG. 8 shows the example embodiment 1300 showing different configuration options from presentation layer for a cloud-based setting. Business Logic Layer 1320 receives the image data from the tenant using the wireless system, which in turn processes the clinical information and gives the output 1330. Such a business layer 1320 can be for (a) cardiovascular application (such as IMT measurement. IMTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease; or f) thyroid disease classification such as benign thyroid or malignant thyroid or Hashimoto Disease Classification, where these applications are the business layers in the three tier architectures such that it can process the B-mode ultrasound or RF-mode ultrasound images. The configuration option is available for choosing the different types of engines such as Scientific Engine Type 1, Scientific Engine Type 2 or Scientific Engine Type 3. Tier 1, 710 can interact with the clinical information 1330 to display the clinical diagnosis on 710, such as iPhone, iPad, Samsung Table, or even laptop, notebook or Desktop-based display devices. The persistence layer process 1340 processes the clinical information 1330 and stores in the persistence layer. This information can also be accessed by Tier-1, 710. Output 1350 is the information which is saved in the cloud or local server. It is important to note that Persistence layer 1340 and clinical data results 1350 are stored in the cloud 1302 while Business Layer 1320 and the clinical information results 1330 are stored in the cloud 1301. Even though the entire data mining application is responding from the presentation layer 710, but the rest of the components are partitioned, in different clouds using wireless operations. Such as business layer 1320 can be for (a) cardiovascular application (such as IMT measurement, IMTV measurement, Plaque Characterization for Symptomatic vs. Asymptomatic classification of plaque, Stroke Risk computation, and monitoring stroke risk); (b) prostate cancer application (such as benign vs. malignant prostate tissue classification or characterization for prostate cancer); (c) ovarian cancer tissue characterization and classification; or (d) thyroid cancer application (such as benign vs. malignant thyroid tissue classification or characterization for thyroid cancer); or (e) classification of liver tissue such as Fatty Liver Disease; or f) thyroid disease classification such as benign thyroid or malignant thyroid or Hashimoto Disease Classification, where these applications are the business layers in the three tier architectures such that it can process the B-mode ultrasound or RF-mode ultrasound images.

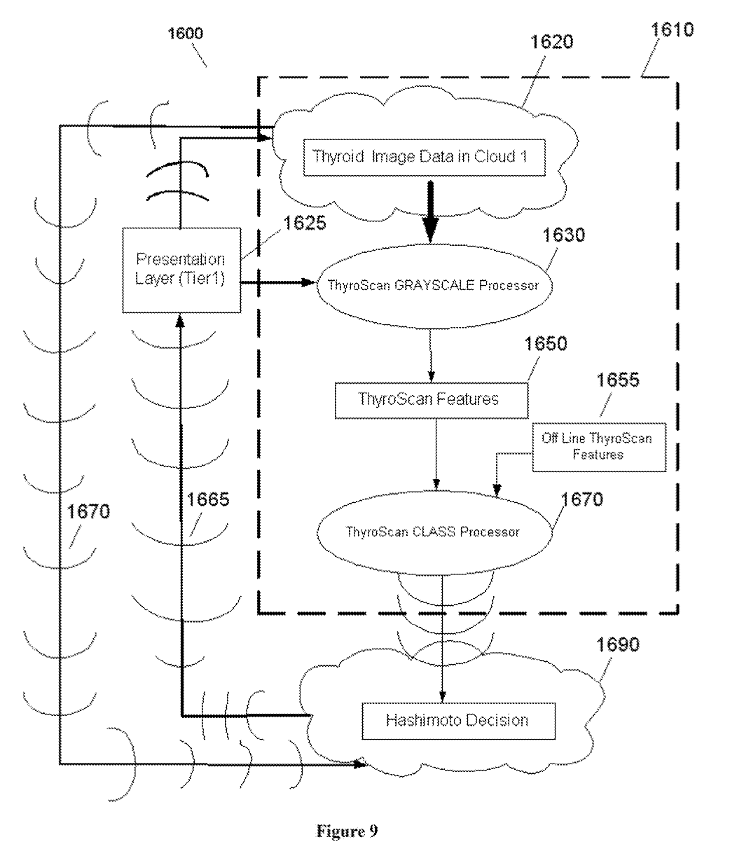

FIG. 9 illustrates an example embodiment 1600 showing the Hashimoto Disease Diagnosis system. Block 1620 receives the image data from health care system in the Cloud 1. Processor 1630 is controlled by block 1625, which is the presentation layer. Block 1630 gives the on tine features of the Thyroid grayscale images. These are fed to the ThyroScan™ class Processor 1670 as part of the Business Layer which yields Hashimoto Binary Decisions as a diagnostic index, and saved in block 1690 in the persistence cloud 2. Block 1625 is a hand-held device which can display the Hashimoto Diagnostic Decision using, the channel 1665. Block 1670 allows saving the image data into the Persistence Layer 1690.

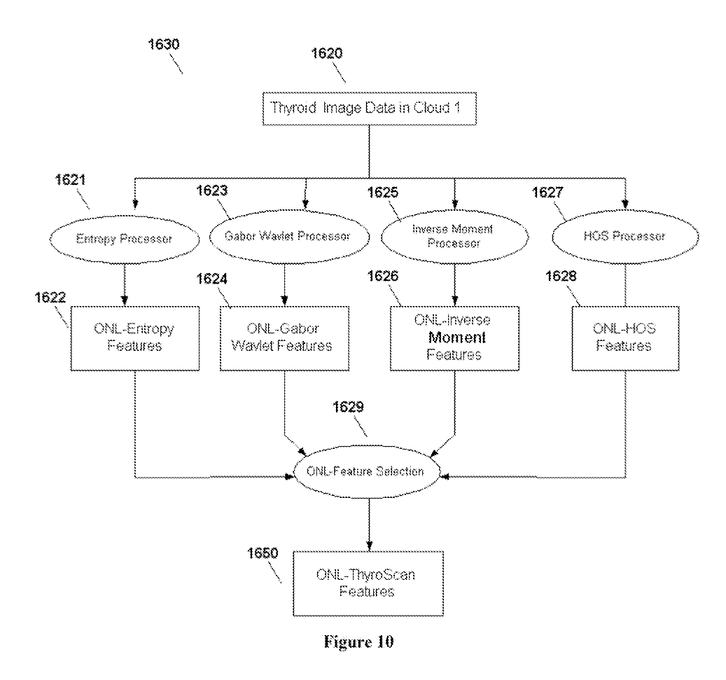

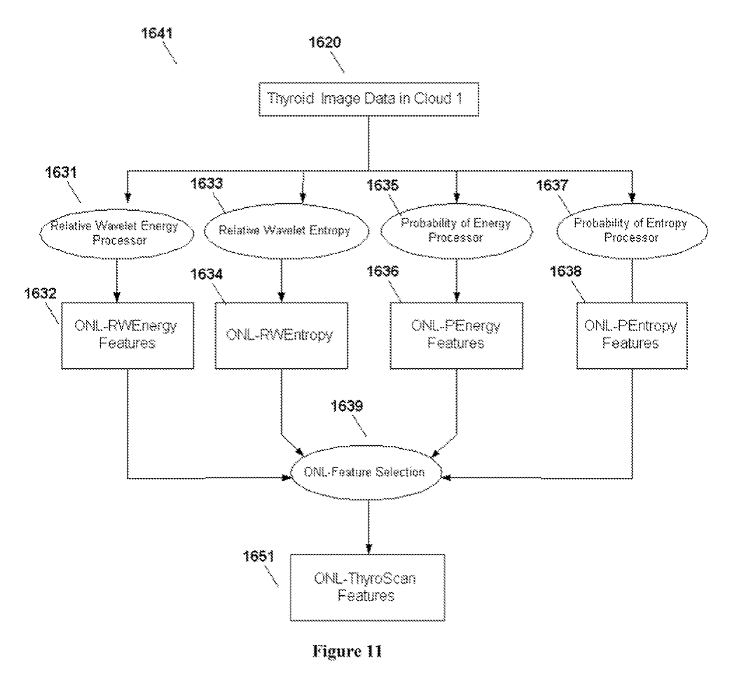

FIG. 10 illustrates an example embodiment 1630 showing the Hashimoto grayscale on line feature extraction system. Block 1621, block 1623, block 1625, and block 1627 use four different kinds of on-line processors for computing four different kinds of features. Block 1621 is an on-line entropy processor which yields the on-line entropy features 1622. Block 1623 is a on-line Gabor Wavelet Processor that computes the on-line Gabor Wavelet Features, Block 1625 is an on-line Inverse Moment Processor and computes the on-line inverse moment features 1626. Block 1627 is a on-line HOS processor which computes the on-line HOS features. The novelty of this set-up is the combination of this feature which constitutes the support in diagnosis of Hashimoto Disease. Block 1629 uses a Feature Selection Processor which finally gives the on line features 1650. The on-line features are fed to the ThyroScan Class Processor 1670 as detailed out in FIG. 9. The block 1610 can be one cloud which feed to the block 1690 in cloud 2. The same concept is applied for the training-based system by the block 1665 as shown in FIG. 11.

FIG. 11 illustrates another example embodiment 1641 showing the Hashimoto grayscale on line feature extraction system. Block 1631, block 1633, block 1625, and block 1637 use four different kinds of on-line processors for computing four different kinds of features. Block 1631 is an on-line relative wavelet energy processor which yields the on-line relative wavelet energy features 1632. Block 1633 is relative wavelet entropy Processor that computes the on-line relative entropy features 1634. Block 1635 is a probability of energy processor which yields online probability of energy features 1636. Block 1637 is an on-line probability of entropy processor which computes the on-line probability of entropy features. The novelty of this set-up is the combination of this feature which constitutes the support in diagnosis of Hashimoto Disease. Block 1639 uses a Feature Selection Processor which finally gives the on line features 1651. The on-line features are fed to the ThyroScan Class Processor 1670 as detailed out in FIG. 9. The block 1610 can be one cloud which feed to the block 1690 in cloud 2. The same concept is applied for the training-based system by the block 1665 as shown in FIG. 11.

### Stationary Wavelet Transform (SWT) for Feature Extraction

Wavelet transform captures both the spatial and frequency information of a signal. Discrete Wavelet Transform (DWT) uses filter banks composed from finite impulse response filters to decompose signals into low and high pass components. The low pass component contains information about slow varying signal characteristics, and the high pass component contains information about sudden changes in the signal. DWT, however, is not a time-invariant transform. The translation invariance of DWT can be restored by using Stationary Wavelet Transform.

A 2D sub-band transform with three levels of decomposition. When low pass filtering, using filter g[n] is applied to both the rows and columns of the image, the LL coefficients are obtained which are called the approximation coefficients ‘A’. These coefficients are representative of the total energy in the images. When low pass filtering is applied to the rows, and high pass filtering using filer h[n] is applied to the column values, the resultant HL coefficients contain the vertical details of the image ‘V’ Row-wise high pass filtering and column-wise low pass filtering result in the LH coefficients, which contain the horizontal details of the image ‘H’. High pass filtering of both row and column values results in the finest-scale HH coefficients, which contain the diagonal details of the image D. Decomposition is further performed on sub-band LL to attain the next coarser scale of wavelet coefficients. The input approximation coefficients cAⱼ and the results for level j+l. In this application, we first converted the image to grayscale range of [0, 255] and then applied SWT using rhio3.1 as the mother wavelet.

After obtaining, the wavelet coefficients at each level of the three-level SWT decomposition, we determined the following features for each of the ten subsets of coefficients: (a) Relative Wavelet Energy (RWEng); (b) Relative Wavelet Entropy (RWEnt); (c) Probability of Energy (PEng), and (d) Probability of Entropy (Pent). Energy probability distribution in scales is the relative wavelet energy. Relative wavelet entropy tells how similar a probability distribution pⱼ is with respect to another probability distribution qⱼ referenced. In the following, sample equations. Eng_{N}^{a} indicates the energy of the approximation coefficients cA obtained at level N. Eng_{N}^{h} indicates the energy of the horizontal detail coefficients cD^{h} obtained at level N. Eng_{N}^{v} indicates the energy of the vertical detail coefficients cD^{v} obtained at level N. Eng_{N}^{d} indicates the energy of the diagonal detail coefficients cD^{d} obtained at level N. Similar definitions hold true for the other terms used in the equations.

$$\begin{matrix}{RWEng\_cA}_{1} \text{:} {RWEng}_{1}^{a} = \frac{{Eng}_{1}^{a}}{{\sum }_{N} {Eng}_{N}^{a} + {\sum }_{N} {Eng}_{N}^{d} + {\sum }_{N} {Eng}_{N}^{v} + {\sum }_{N} {Eng}_{N}^{h}} & ( 1 ) \\ {PEng\_cA}_{2} \text{:} {PEng}_{2}^{a} = \frac{{Eng}_{2}^{a}}{{\sum }_{N} {Eng}_{N}^{a} + {Eng}_{2}^{d} + {Eng}_{2}^{h} + {Eng}_{2}^{v}} & ( 2 ) \\ {RWEnt\_cA}_{1} \text{:} {RWEnt}_{1}^{a} = \frac{{Ent}_{1}^{a}}{{\sum }_{N} {Ent}_{N}^{a} + {\sum }_{N} {Ent}_{N}^{d} + {\sum }_{N} {Ent}_{N}^{v} + {\sum }_{N} {Ent}_{N}^{h}} & ( 3 ) \\ {RWEnt\_cH}_{2} \text{:} {RWEnt}_{2}^{h} = \frac{{Ent}_{2}^{h}}{{\sum }_{N} {Ent}_{N}^{a} + {\sum }_{N} {Ent}_{N}^{d} + {\sum }_{N} {Ent}_{N}^{v} + {\sum }_{N} {Ent}_{N}^{h}} & ( 4 ) \\ {RWEnt\_cV}_{2} \text{:} {RWEnt}_{2}^{v} = \frac{{Ent}_{2}^{v}}{{\sum }_{N} {Ent}_{N}^{a} + {\sum }_{N} {Ent}_{N}^{d} + {\sum }_{N} {Ent}_{N}^{v} + {\sum }_{N} {Ent}_{N}^{h}} & ( 5 ) \\ RWEnt\_A \text{:} {RWEnt}_{N}^{a} = \frac{{\sum }_{N} {Ent}_{N}^{a}}{{\sum }_{N} {Ent}_{N}^{a} + {\sum }_{N} {Ent}_{N}^{d} + {\sum }_{N} {Ent}_{N}^{v} + {\sum }_{N} {Ent}_{N}^{h}} & ( 6 ) \\ {PEnt\_cA}_{2} \text{:} {PEnt}_{2}^{a} = \frac{{Ent}_{2}^{a}}{{\sum }_{N} {Ent}_{N}^{a} + {Ent}_{N}^{d} + {Ent}_{N}^{h} + {Ent}_{N}^{v}} & ( 7 )\end{matrix}$$

where Eng_{N}^{a}=Σₖ|cA_{N}(k)|²; Ent_{N}¹=−ΣₖcA_{N}²(k)log(cA_{N}²(k))
where N is the number of levels of decomposition, taken as 3; and k is the number of coefficients at each decomposition level.

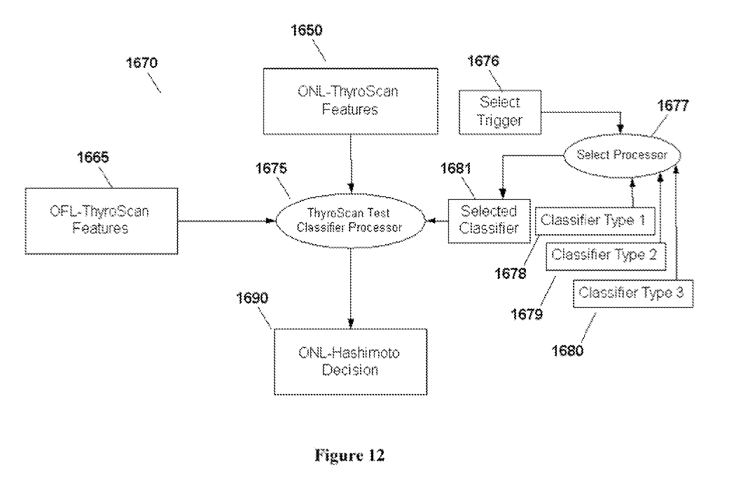

FIG. 12 shows the example embodiment 1670 showing the table concept for an image-based data mining application using the Cloud Concept to Hashimoto Disease Diagnosis utilizing the ThyroScan Test Classifier. Block 1650 receives the online grayscale features. Block 1677 shows the select processor for selection of the type of the classifier, given three sets of classifiers: 1681, 1679 and 1680. Select Trigger 1676 is sent to the Select Processor 1677 and corresponding Classifier Type is selected out of 1681, 1679 and 1680 and the output 1685 is fed to the block 1675 which is used for classification of the online feature of the grayscale thyroid scan 1650. Note that the block 1675 uses off-line Hashimoto features along with the on-line Thyroid Scan features and yields the Hashimoto binary decision if the Thyroid has the Hashimoto Disease or not.

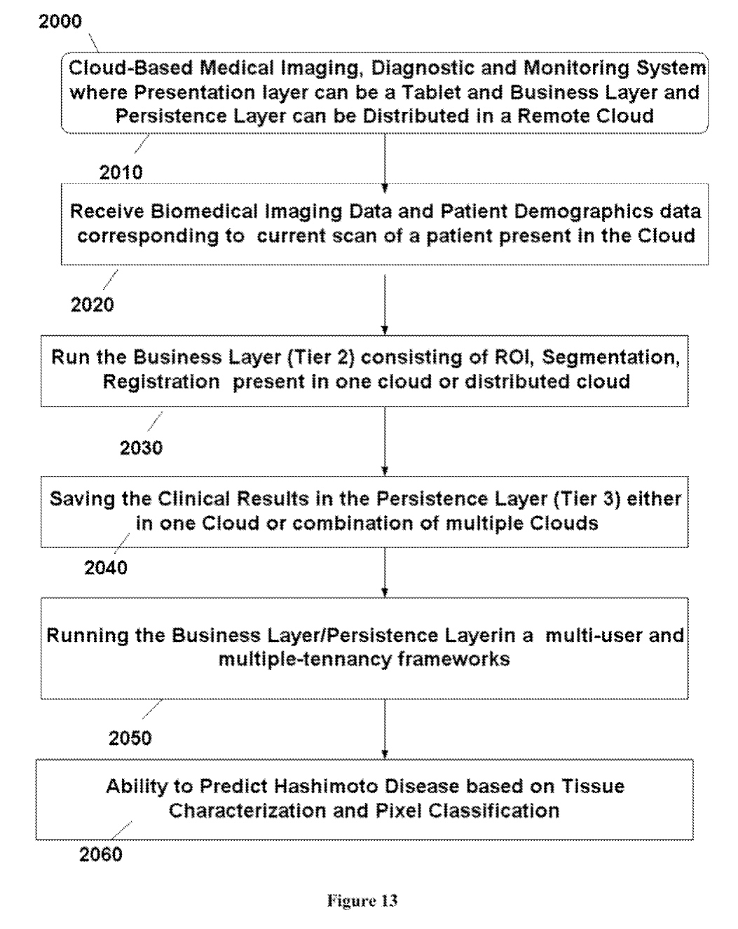

FIG. 13 shows the example embodiment 2000 of the data mining application. Data mining application 2010 using single Clouds or a set of Clouds which consist of Tier-1 as a presentation layer. Tier-2 is the business layer and Tier-3 is the Persistence Layer. The set-up 2010 is used for diagnostic and monitoring application. The Presentation Layer in data mining framework for cardiovascular risk assessment, stroke risk assessment, liver disease assessment, vascular imaging assessment such as IMT measurement using AtheroEdge™, plaque characterization using Atheromatic™, stroke risk assessment using AtheroRisk™, atherosclerosis disease monitoring using Atherometer™, Vessel Analysis using, VesselOmeasure™, fatty liver disease characterization using Symptosis™, tissue characterization for prostate using UroImage™ and Thyroid Disease Diagnosis, particularly Hashimoto Disease Classification and Management. Block 2020 receives the image data from the Cloud for processing. Block 2030 runs the business layer and Block 2040 is the Persistence Layer for the application. Block 2050 is the block where the application can use multiple tenancy-multi use frame work. Block 2060 show the Hashimoto Disease Diagnosis Application using multiple image-based setting such as Ultrasound, MR, CT, or its fusion.

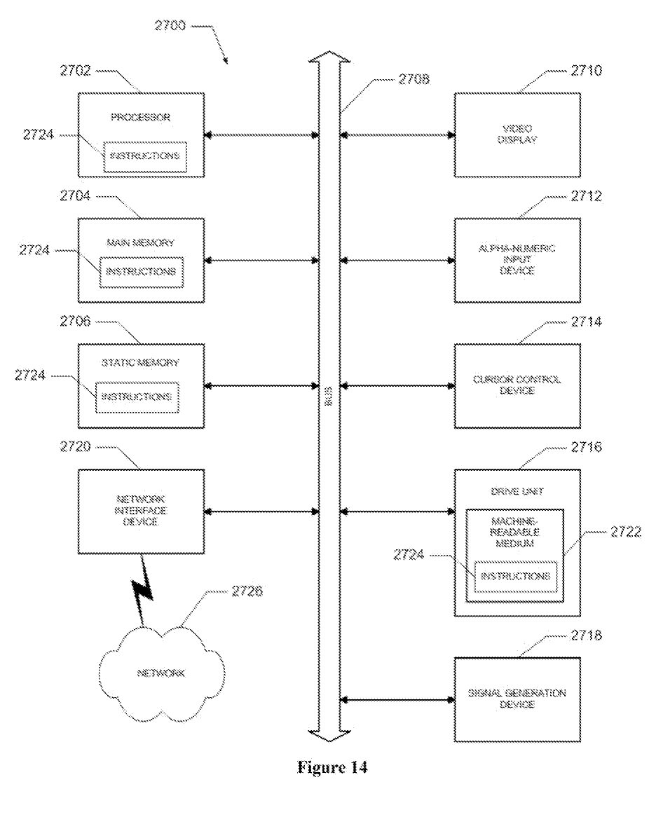

FIG. 14 shows a diagrammatic representation of machine in the example form of a computer system 2700 within which a set of instructions when executed may cause the machine to perform any one or more of the methodologies discussed herein. In alternative embodiments, the machine operates as a standalone device or may be connected (e.g., networked) to other machines. In a networked deployment, the machine may operate in the capacity of as server or a client machine in server-client network environment, or as a peer machine in as peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing as set of instructions (sequential or otherwise) that specify actions to be taken by that machine. Further, while only a single machine is illustrated, the term “machine” can also be taken to include any collection of machines that individually or jointly execute a set (or multiple sets) of instructions to perform any one or more of the methodologies discussed herein.

The example computer system 2700 includes a processor 2702 (e.g., a central processing unit (CPU), a graphics processing unit (GPU), or both), a main memory 2704 and a static memory 2706, which communicate with each other via a bus 2708. The computer system 2700 may further include a video display unit 2710 (e.g., a liquid crystal display (LCD) or a cathode ray tube (CRT)). The computer system 2700 also includes an input device 2712 (e.g., a keyboard), a cursor control device 2714 (e.g., a mouse), a disk drive unit 2716, a signal, generation device 2718 (e.g., a speaker) and a network interface device 2720.

The disk drive unit 2716 includes a machine-readable medium 2722 on which is stored one or more sets of instructions (e.g., software 2724) embodying any one or more of the methodologies or functions described herein. The instructions 2724 may also reside, completely or at least partially, within the main memory 2704, the static, memory 2706, and/or within the processor 2702 during execution thereof by the computer system 2700. The main memory 2704 and the processor 2702 also may constitute machine-readable media. The instructions 2724 may further be transmitted or received over a network 2726 via the network interface device 2720. While the machine-readable medium 2722 is shown in an example embodiment to be a single medium, the term “machine-readable medium” should be taken to include a non-transitory single medium or multiple media (e.g., a centralized or distributed database, and/or associated caches and servers) that store the one or more sets of instructions. The term “machine-readable medium” can also be taken to include any medium that is capable of storing, encoding or carrying a set of instructions for execution by the machine and that cause the machine to perform any one or more of the methodologies of the various embodiments, or that is capable of storing, encoding or carrying data structures utilized by or associated with such a set of instructions. The term “machine-readable medium” can accordingly be taken to include, but not be limited to, solid-state memories, optical media, and magnetic media.

The Abstract of the Disclosure is provided to comply with 17 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A computer-implemented method comprising:
receiving image data on a mobile presentation device, such as hand-held device having a display screen, from a current image of a patient record stored in a network cloud;
using a data processor in data communication with a tier-2 business layer containing a data mining application in the network cloud;
using the data processor in data communication with the tier-2 business layer containing an automated data mining application in the network cloud with several configurations for creating multiple business layers or a fusion of multiple business layers;
using the data processor in data communication with a tier-3 persistence layer containing an automated data mining application in network communication with the tier-2 business layer;
using the data processor in data communication with a tier-1 presentation layer for displaying processed results computed by the automated data mining application and computed using a combination of the tier-2 business layer and the tier-3 persistence layer, the tier-1 presentation layer being configured to communicate with the tier-2 business layer and the tier-3 persistence layer of a three tier architecture;
using a combination of the tier-2 business layer and the tier-3 persistence layer with a combination of a training classifier and a testing classifier; and
using the testing classifier as an online system for computing a binary diagnostic index for Hashimoto's Thyroiditis (HT) Disease.

2. The method as claimed in claim 1 which can be used for diagnosis or monitoring of Hashimoto's Thyroiditis (HT) Disease.

3. The method as claimed in claim 1 which can be used for diagnosis or monitoring of benign vs. malignant Thyroid cancer index (ThyroScan™).

4. The method as claimed in claim 1 where the tier-2 business layer can be an ultrasound B-mode data or an RF mode ultrasound data set for Hashimoto's Thyroiditis HT diagnosis.

5. The method as claimed in claim 1 where the tier-2 business layer comprises an online processor for computing of four grayscale features: Entropy-based feature, Gabor Wavelet-based Feature, Inverse Moment-based feature, and Higher Order Spectra Features.

6. The method as claimed in claim 1 where the tier-2 business layer comprises an online processor for computing of four grayscale features: Set 1: Relative Wavelet Entropy, Relative Wavelet Energy, Probability Energy, Probability Entropy OR Set 2: Relative Wavelet Entropy, Relative Wavelet Energy, Probability Energy, Probability Entropy.

7. The method as claimed in claim 1 where the tier-2 business layer comprises an online processor for computing the online features such as: Set 1: Entropy-based feature, Gabor Wavelet-based Feature, inverse Moment-based feature, and Higher Order Spectra Features OR Set 2: Relative Wavelet Entropy, Relative Wavelet Energy, Probability Energy, Probability Entropy; and further using a feature selector for selecting the best combination of features and further using these features in combination of off-line Thyroid vectors for diagnosis HT.

8. The method as claimed in claim 1 where the set-up of the tier-2 business layer can have several configurations controlled by the tier-1 presentation layer, the configurations can use different classifiers for HT diagnosis from the classifier group: SVM, KNN, and BPPNN.

9. The method as claimed in claim 7 where the off-line Thyroid vectors uses the same set of four features: Set 1: Entropy-based feature, Gabor Wavelet-based Feature, Inverse Moment-based feature, and Higher Order Spectra Features OR Set 2: Set 2: Relative Wavelet Entropy, Relative Wavelet Energy, Probability Energy, Probability Entropy;

10. The method as claimed in claim 1 where the tier-2 business layer can receive the MR data.

11. The method as claimed in claim 1 where the tier-2 business layer can be a CT data.

12. The method as claimed in claim 1 where the set-up of tier-1 presentation layer includes a hand-held device, a laptop or notebook or a desktop or an iPhone or a tablet and receives data from the tier-2 business layer and the tier-3 persistence layer using the controls of the tier-1 presentation layer.

13. The method as claimed in claim 1 where the set-up of the tier-2 business layer can be in one network cloud and the tier-3 persistence layer can be in the same or another network cloud, in a distributed cloud architecture by splitting the different tiers of the three tier architecture for computing a diagnostic index for benign vs. malignant tissue for thyroid cancer diagnosis, and diagnosis of HT.

14. The method as claimed in claim 1 where the set-up uses a wireless system for data transfer between the tier-1 presentation layer and the tier-2 business layer and vice-versa.

15. The method as claimed in claim 1 where the set-up uses a wireless system for data transfer between the tier-1 presentation layer and the tier-3 persistence layers and vice-versa.

16. The method as claimed in claim 6 where the tier-2 business layer can be utilize any 2D or 3D segmentation engine for computation of a region of interest (ROI) and then compute the grayscale features such as Entropy-based feature, Gabor Wavelet-based Feature, Inverse Moment-based feature, and Higher Order Spectra Features in this region of interest.

17. The method as claimed in claim 16 where the tier-2 business layer can be utilize any 2D or 3D segmentation engine for computation of as region of interest (ROI), where the region of interest can be computed automatically or semi-automatically.

18. The method as claimed in claim 16 where the tier-2 business layer can be utilize any 2D or 3D segmentation engine for computation of a region of interest (ROI), where the region of interest can be computed using a trained atlas.

19. The method as claimed in claim 16 where the tier-2 business layer can be utilize any 2D or 3D segmentation engine for computation of a region of interest (ROI) and then compute the grayscale features such as Entropy-based feature, Gabor Wavelet-based Feature, Inverse Moment-based feature, and Higher Order Spectra Features in this region of interest and followed by feature selection system for selecting the best features.

20. The method as claimed in claim 1 where the tier-2 business layer can utilize thyroid image data from the left lobe or right lobe or can be combined using left and right lobe.
